(19) United States
(12) Patent Application Publication
(10) Pub. No.: US 2012/0035721 A1
(43) Pub. Date: Feb. 9, 2012

(54) TEMPORARY SUB-VALVULAR CHECK VALVE

(75) Inventor: Ivan Vesely, Larkspur, CO (US)

(73) Assignee: ValveXchange, Inc., Aurora, CO (US)

(21) Appl. No.: 13/206,340

(22) Filed: Aug. 9, 2011

Related U.S. Application Data
(60) Provisional application No. 61/371,911, filed on Aug. 9, 2010.

(51) Int. Cl. A61F 2/24

(52) U.S. Cl. 623/2.36

(57) ABSTRACT

A temporary subvalvular check valve has a collar and an expandable seal structure connected by pivotable struts that together support flexible leaflets. The check valve can be introduced along a tool shaft and positioned in a chamber or vasculature by expanding the seal structure against an adjacent wall. Cardiac function is augmented during valve procedures, such as valve excision, valve implantation, or valve leaflet replacement, by placing the temporary check valve just upstream of the valve being treated. The temporary check valve is collapsible so that it can be inserted through a small incision or port in the apex of the heart or through the aorta, into the ventricular cavity. Such a system thus does not require arrest or pacing of the heart and will allow such valve repair or replacement procedures to be done without concern for time or compromise to the patients' physiology.

### CROSS REFERENCE TO RELATED APPLICATIONS

This application claims the benefit of priority pursuant to 35 U.S.C. §119(e) of U.S. provisional application no. 61/371,911 filed 9 Aug. 2010 entitled “Temporary sub-valvular check valve,” which is hereby incorporated herein by reference in its entirety.

### FIELD OF TECHNOLOGY

The present disclosure relates generally to a system for performing procedures on native or prosthetic heart valves, and more particularly to the servicing, repair, or replacement of these devices without requiring cardiopulmonary bypass.

### BACKGROUND

The demographics of patients suffering valvular disease are broad and the treatment modalities for each are complex. Historically, patients younger than 65 years of age have been prescribed mechanical valves and those older receive bioprosthetic valves. These prosthetic valves eventually wear out and need to be replaced with a new, functional device.

Replacement of native or prosthetic valves has traditionally required open-chest, open-heart surgery in which the patient is placed on cardiopulmonary bypass and the heart stopped and restarted again after the surgery is completed. Cardiopulmonary bypass, however, is associated with both short-term and long-term complications, such as cognitive impairment. This apparently results from the low-grade damage that occurs to the blood cells as they pass through the heart-lung machine. Although not completely understood, the process of subjecting blood constituents to pumps and oxygenators leads to the formation of small clots which then cause micro-strokes when introduced back into the patient.

Performing off-pump, beating-heart surgery has been a great challenge and an important objective for surgeons for many decades; the most successful today is the surgical repair of atherosclerotic lesions in the coronary arteries. Off-pump Coronary Artery Bypass Surgery (OP-CABG) is now considered a huge advantage for patients that require Coronary Artery Bypass. Although the chest is opened to allow access to the surface of the heart, the heart continues to pump as the blood vessels that feed blood to the heart are repaired and bypassed.

Surgery on other components of the heart, such as the valves of the heart, is more difficult. This is because the valves are inside the heart and opening the heart to expose them cannot be done with the heart pumping blood. The primary candidate technology for treating heart valves without stopping the heart and opening it up is the use of catheter-implantable valves. These valves are delivered through a catheter passed up through the aorta or the aortic arch, or through the apex of the heart into the ventricular cavity. These valves consist of tissue leaflets mounted on a frame that is expanded and anchored in the vicinity of the existing diseased native valve.

Transcatheter Aortic Valve Implantation (TAVI) has become very popular because it avoids the potential complications of opening up the chest and placing the patient on cardiopulmonary bypass. This new technology is thus used on the very old or sick patients that have a high risk of dying if they were to undergo conventional open-heart surgery on cardiopulmonary bypass.

The challenge in performing TAVI is the need to perform the procedure quickly. During the procedure itself, the native heart valve is crushed against the sides of the aorta, and during that process, the patient is essentially without a valve and thus without normal cardiac output flow. Moreover, many current generation transcatheter valves are expanded and seated in place by way of a balloon which occludes the aorta, essentially preventing any ejection of blood from the heart. To enable the balloon and the associated transcatheter valve from being ejected out of the heart, the physician rapidly paces the heart, dramatically reducing its contractions and preventing the pumping of blood. This is not an ideal situation for the patient, particularly if they are already ill and compromised from the underlying valvular disease.

Another approach has been to augment the native valve with a temporary valve to augment the pumping of blood while the native valve is in the process of being repaired, excised, or replaced with a prosthetic valve.

Prior temporary check valves have been placed in the ascending or descending aorta. This is not an ideal location, since the temporary valve is downstream from the coronary arteries and does not function in the appropriate manner during diastole to help in the filling of the coronary arties, as with the native aortic valve.

There is also a situation when the native valve has already been replaced with a prosthetic device, and that device itself needs replacement. One such technology is the exchangeable valve concept (disclosed, for example, in U.S. Pat. No. 7,011,681 B2), in which the old, worn-out leaflet set may be pulled off the base of the valve and replaced with a new one. Such a procedure may also benefit from the use of an appropriate temporary check valve.

The information included in this Background section of the specification, including any references cited herein and any description or discussion thereof, is included for technical reference purposes only and is not to be regarded subject matter by which the scope of the invention is to be bound.

### SUMMARY

The technology presented herein is a system for augmenting cardiac function during valve procedures, such as valve excision, valve implantation, or valve leaflet replacement, by placing a temporary check valve just upstream of the valve being treated. The temporary check valve is collapsible so that it can be inserted through a small incision or port in the apex of the heart or through the aorta, into the ventricular cavity. Such a system thus does not require arrest or pacing of the heart and will allow such valve repair or replacement procedures to be done without concern for time or compromise to the patients' physiology.

As suggested, a better location for the temporary check valve is upstream of the native aortic valve or essentially inside the ventricle, yet in intimate contact with the aortic outflow tract. Having the temporary check valve upstream of the coronary arteries, and upstream of the native valve, will enable procedures to take place on the native aortic valve and still facilitate the proper ejection of blood from the ventricle and the filling of the coronary arteries in a physiologically appropriate manner.

A further application of the temporary check valve is in conjunction with the placement of a valve-supporting frame without the leaflets. The valve supporting frame can be incrementally dilated until it fits snugly in the patient's aortic root and the appropriately sized leaflets that fit into that frame can then be delivered. During the positioning and expansion of the frame, the patient may be without a fully functioning valve. The temporary check valve may thus help provide continuous cardiac output until the final leaflet set is delivered onto the valve frame.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1A is a schematic side isometric view of a temporary check valve mounted on a tool shaft passed into the interior of the heart.

FIG. 1B is a side elevation view in cross section of the temporary check valve of FIG. 1A as indicated by line 1B-1B in FIG. 2.

FIG. 2 is top plan view showing the outflow end of the temporary check valve of FIG. 1A, with one segment of the mesh removed.

FIG. 3 is a top isometric view of a schematic drawing of the temporary check valve shown in FIG. 1A.

FIG. 4 is an isometric view of the temporary check valve of FIG. 1A positioned against the underside of a bioprosthetic valve with exchangeable leaflets.

FIG. 5 is a bottom isometric view of the temporary check valve of FIG. 1A positioned against the sewing cuff of a bioprosthetic valve as in FIG. 4. In this view, one segment of the mesh is removed for clarity.

FIG. 6 is a bottom perspective view of the temporary check valve of FIG. 1A in a collapsed configuration. The mesh is removed for clarity.

FIG. 7 is a bottom isometric view of the temporary check valve of FIG. 1A positioned at an intermediate location along the length of the tool shaft. The leaflets and mesh are removed for clarity.

FIG. 8 is a bottom isometric view of the temporary check valve of FIG. 1A with one segment of the mesh removed to show the leaflets collapsed around the outside surface of the central tool shaft.

FIG. 9A is a top isometric view of the temporary check vale of FIG. 1A in an open flow configuration. In this view, one of the mesh segments is removed for clarity.

FIG. 9B is a bottom isometric view of the temporary check vale of FIG. 1A in an open flow configuration. In this view, one of the mesh segments is removed for clarity.

FIG. 9C is a bottom isometric view of the temporary check valve of FIG. 1A in a fully closed configuration. In this view, one of the mesh segments is removed for clarity.

FIG. 10 is a bottom isometric view of the temporary check valve of FIG. 1A positioned below a valve frame of a two-part valve system. In this view, one of the mesh segments is removed for clarity.

FIG. 11 is an isometric view of the temporary check valve of FIG. 1A shown in vivo placed in the ventricle below the aortic valve.

### DETAILED DESCRIPTION

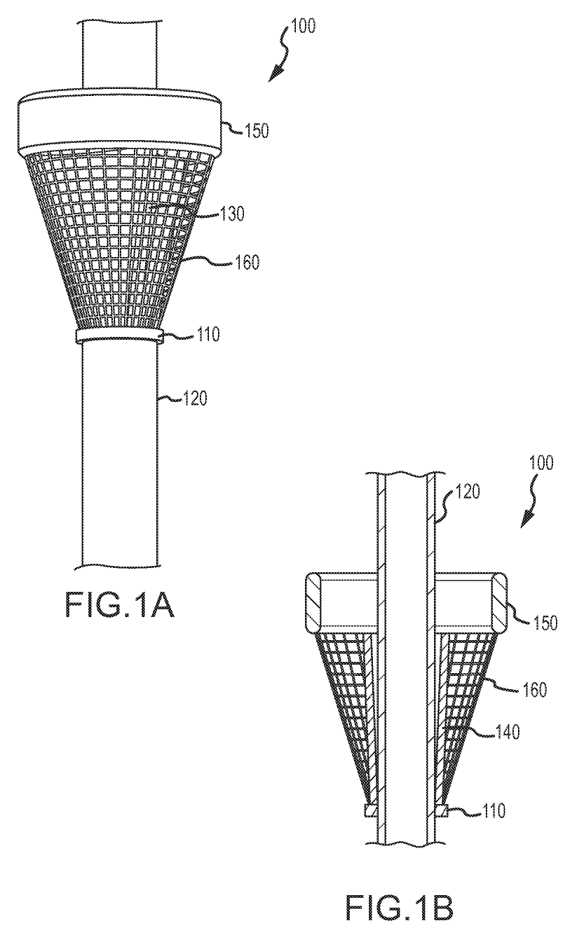

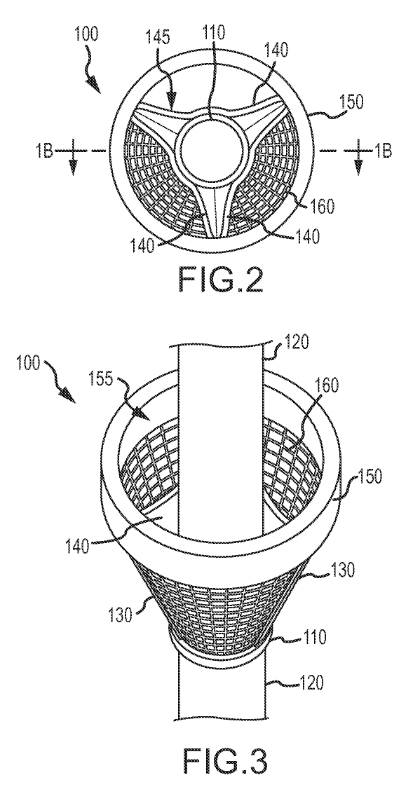

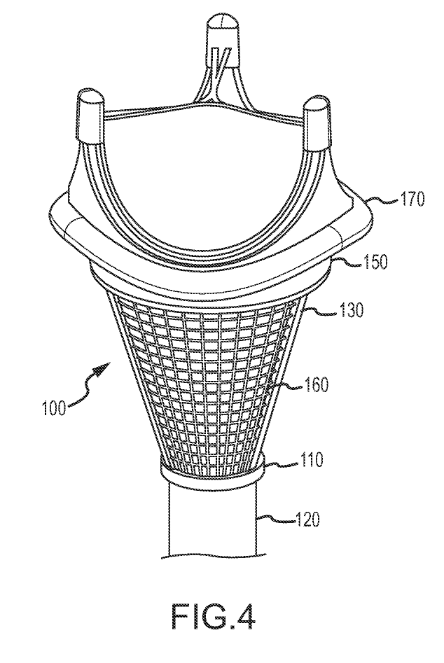

A temporary check valve that can be positioned below the existing valve, e.g., below the aortic valve 220, and augment the function of the original valve is disclosed herein in conjunction with the accompanying FIGS. 1A-11.

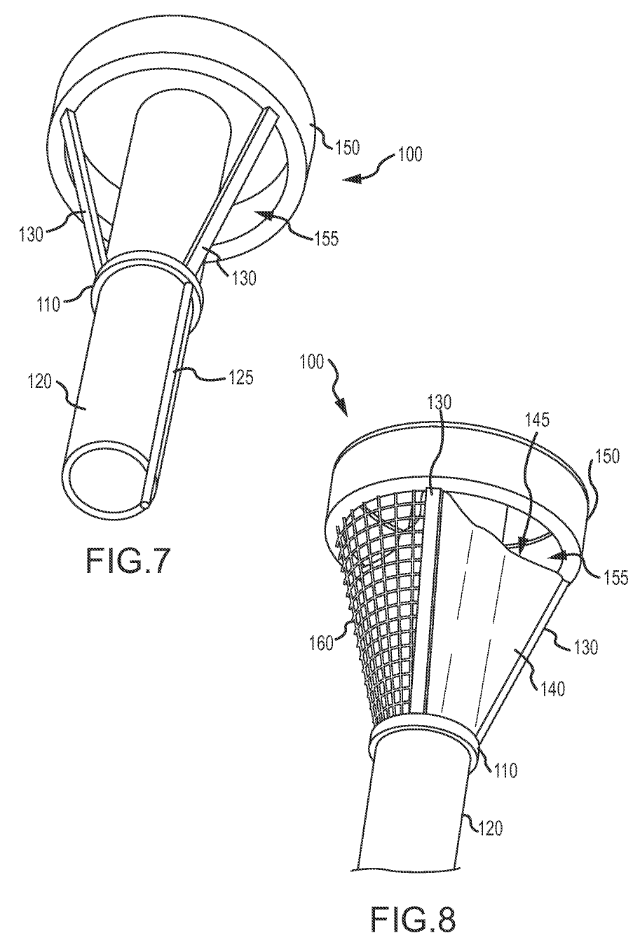

According to a first implementation, and as can be seen in FIGS. 1A-3, and others, a temporary check valve 100 is composed of a sliding collar 110 that is positioned along the length of an insertion and guide tool 120, e.g., a hollow tube, shaft, or catheter. The sliding collar can be repositioned along the shaft with a control structure, for example, sliders or pull wires 125 (as shown in FIG. 7). The tool shaft 120 may facilitate the insertion or action of additional tools and may thus move along its axis further into the heart, independent of the check valve 100.

As shown in FIGS. 1A-3 and 7-8, and others, one or more (preferably three) struts 130 that support three leaflets 140 at their side edges project from the sliding collar 110 from one end of each. The struts 130 may be made of a harder material than the leaflets 140, and are hinged at their attachment to the sliding collar 110 so that they can pivot radially outward. At the distal end of each of the struts 130 is an annular seal structure 150 that can expand and contract and seal against the interior walls of the vessels or chambers into which the tool shaft 120 and the temporary check valve 100 are inserted. As the seal structure 150 is expanded, the struts 130 pivot outward and the valve leaflets 140 are thereby expanded into position. The sliding collar 110 is held in a fixed position at a fixed distance proximal to the seal structure 150 by the fixed length struts 130. The tool shaft 120 is free to move distally and proximally through the temporary check valve 100 within the sliding collar 110. The side edges of the leaflets 140 are connected to the struts 130 and the narrow proximal base of each of the leaflets 140 is connected to the sliding collar 110. The leaflets 140 thus are free to move at their distal unsupported edges 145.

The annular seal structure 150, which is attached to the struts 130 at their distal ends, is generally circular in shape and can be activated to increase or decrease its circumference as necessary. A toroidal balloon is one possible embodiment of such an annular seal structure 150. The balloon can be inflated with saline, as is done in other balloon applications. A tube for inflating the balloon is not shown in the figures, but can run through the inside of one of the struts 130, or can be positioned along side one of the struts 130, and can run along the outside or inside the tool shaft 120, or can be incorporated into the wall material of the tool shaft 120. As is customary in the field, these components may be made of molded or extruded plastic.

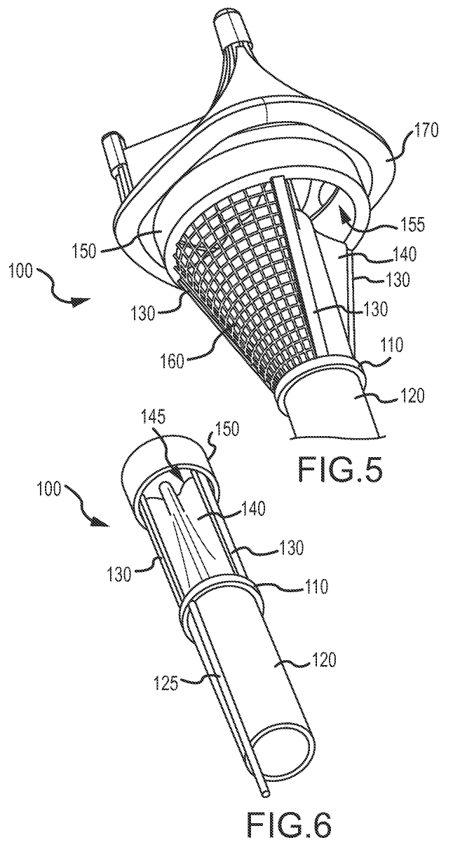

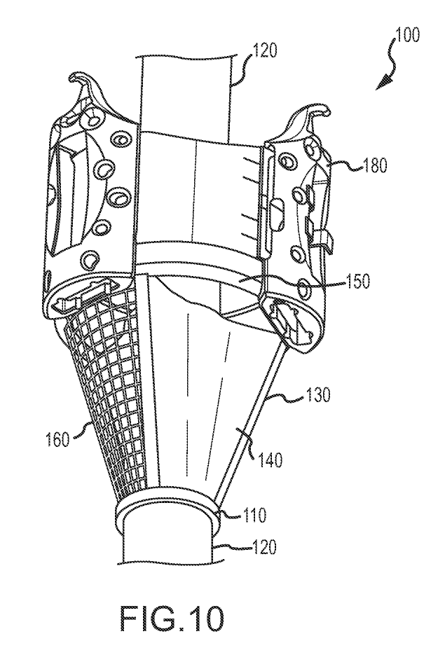

In one implementation, the seal structure 150 may be designed to mate with the underside of an existing bioprosthetic valve 170, e.g., as shown in FIGS. 4 and 5, as well as against the inner surface of a blood vessel or areas of the heart 200 such as in or near the aortic root to displace the native aortic valve leaflets 220. FIG. 10 shows a two-part bioprosthetic valve in which the valve frame 180 can be inserted first, dilated until it fits snugly in the valve position, and the appropriately sized leaflet set may then be snapped in place. This view shows the valve frame 180 without the leaflets in place and with the temporary check valve 100 in position firmly underneath the valve frame 180.

An appropriate number of segments of a collapsible mesh 160 are also attached to the struts 130. The mesh 160 becomes substantially tight as the leaflets 140 are expanded and the struts 130 articulate outward by the expansion of the seal structure 150 which may be dilated in conjunction with or independently of valve frame 180. When the temporary check valve 100 is closed, the leaflets 140 lean against this mesh 160 and are supported by it to form a seal against back pressure. Both the leaflets 140 and the supporting mesh 160 may be generally conical when fully expanded, as shown in the figures, but are not limited to this configuration. A generally conical shape is assumed for the following further description. When the temporary check valve 100 is closed, the leaflets 140 are pushed against the mesh 160 and mesh 160 holds the leaflets 140 in their conical shape. The free edge 145 of the leaflets 140 projects upwards past the mesh 160, so that the leaflets 140 overlap with the inside surface 155 of the annular seal structure 150. The free edge 145 of the leaflets 140 thus seals against the inner surface 155 of the annular seal structure 150 so that there is no leakage of fluid back through the temporary check valve 100 into the chamber 210 of the heart 200 during ventricular diastole.

The presence of the mesh is desirable in that it allows the leaflets to be made very thin and collapsible, such as thin sheets of plastic that can easily fold up, as shown in FIG. 6. Without the mesh 160, the leaflets 140 would need to be stiffer and stronger in order to bear the force of the fluid pressure, which is possible with materials such as thin, highly flexible metal such as Nitinol. The mesh 160 can be made from any appropriate material, such as plastic, fabric, string or could also be made of highly flexible metal, such as Nitinol. It is attached to the struts by way of sutures or pins, or can be overmolded as part of the other plastic components.

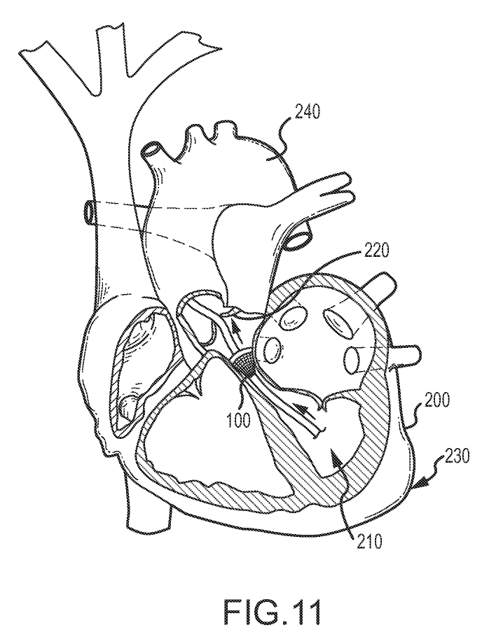

One method of introducing the temporary check valve 100 into the ventricular cavity 210 is through a puncture in the ventricle 210 near the apex 230 of the heart 200 as depicted in FIG. 11. The temporary check valve 100 is held collapsed when the annular seal structure 150 is deflated or otherwise collapsed (as shown in FIG. 6). That minimizes the diameter of the seal structure 150 and brings the struts 130 against the surface of the tool shaft. When the struts 130 are collapsed against the body of the tool shaft 120 and the leaflets 140 are appropriately wrinkled up and folded.

The collapsed temporary check valve 100 may be positioned at the end of the tool shaft 120 for insertion or, alternatively, the tool shaft 120 may be initially inserted and the temporary check valve 100 may be placed about the outer diameter of the tool shaft 120 and slid along the tool shaft 120 until it is in an appropriate location for deployment. A pull wire 125 or rod or additional concentric shaft may be connected to the bottom edge of the sliding collar 110 to control and slide the temporary check valve 100 along the length of the tool shaft 120.

The temporary check valve 100 is then positioned just below the existing valve 220 inside the heart 200, and the seal structure 150 is dilated or inflated (e.g., using a toroidal balloon) until it seals against the walls of the ventricular chamber, or the inner or under-surface of an existing prosthetic valve, as shown in FIGS. 4, 5 and 10. If the seal structure 150 of the temporary check valve 100 is made of an elastically-expanding, toroidal balloon, its collapsed shape is similar to that shown in FIG. 6—a similar toroidal balloon with a smaller inside and outside diameter. If the seal is made from a less-elastic, non-inflatable material, then the seal structure 150 may be folded up and wrinkled as its diameter is reduced, much like the leaflets 140 of the temporary check valve 140 wrinkle up as the struts 130 are folded. If the seal is made from helically wound up material such as thin metal, it may be unwound in position expanding its diameter to the desired dimension.

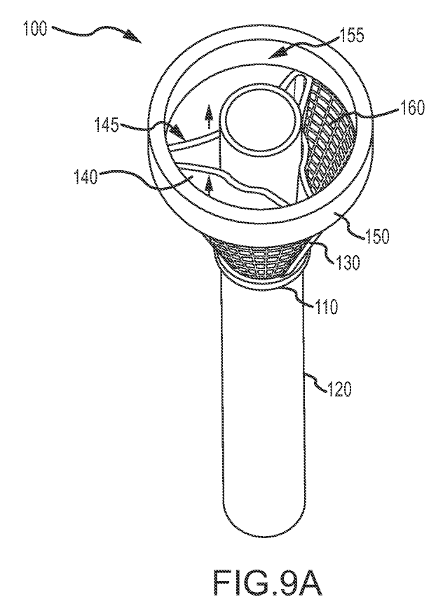

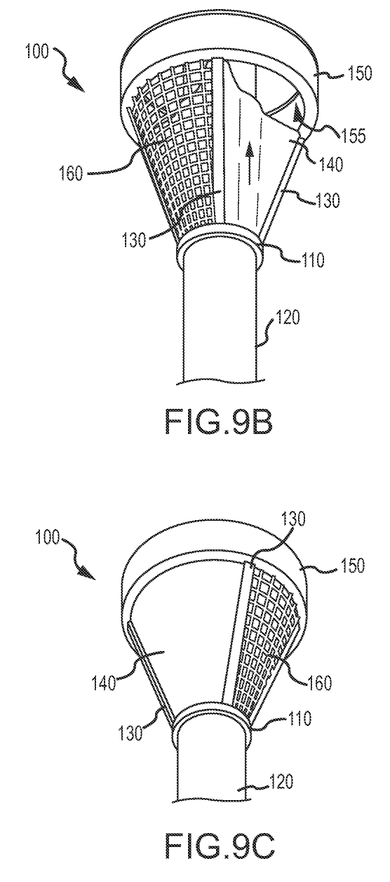

In normal function, all three leaflets 140 are either all open or all closed. During systole, the ventricle contracts and forces blood toward the temporary check valve 100. The leaflets 140 collapse against the tool shaft 120 and blood flows through the mesh 160, past the leaflets 140, and between the tool shaft 120 and the seal structure 150. During diastole, the leaflets 140 are forced proximally against the mesh 160 and seal against the inner surface 155 of the seal structure 150, thus preventing backflow of blood into the ventricle 210 (or other vessel or anatomic area in which the temporary check valve 100 may be positioned). FIGS. 9A and 9B are schematic views showing how blood passes through the temporary check valve 100 when the leaflets 140 are open (i.e., collapsed). In these views, one of the mesh segments is removed for clarity. The arrows show the direction of blood flow. FIG. 9C depicts the leaflets 140 in the closed position with the leaflets 140 pressed against the mesh segments 160 to prevent backflow.

Once the temporary check valve 100 is in place, additional tools can be passed through the central lumen of the tool shaft 120 over which the temporary check valve 100 is positioned, to perform the necessary procedures on the native valve 220 or prosthetic valve 170 without compromising the cardiac output function of the heart 200 or interfering with the normal filling of the coronary arteries. Provisions may be made so that any tool inserted through this hollow tool shaft 120 is appropriately sealed to prevent blood from leaking out of the heart 200 through the hollow tool shaft 120.

In a second implementation, the hollow tool shaft 120 on which the temporary check valve 100 is mounted may be a catheter that is passed through the aorta 240 from downstream of the native valve 220, through the native valve 220, and positioned below the native valve 220 as oppose to being delivered and positioned through the apex 230 of the heart 200.

In accordance with a third implementation, the annular seal structure 150 may be fabricated from a solid structure, for example, an elastic hoop of wire that is self expanding once it is positioned on the interior of a chamber or vessel wall. In one embodiment, the wire hoop may be formed of a shape memory material, e.g., Nitinol. The solid structure may have sufficient elasticity to push against the inner surface of the wall and make the necessary seal, similar to the inflated balloon. The solid structure may also contain an appropriately compliant covering material to properly deform and make contact with the wall of the ventricle chamber or vessel within which it is inserted. A helically wound configuration, unwound to expand and push against the inner surface of the wall can also be used as previously described.

In a fourth implementation, the seal structure may be manually compressed and folded by the action of the struts 130. If the struts 130 are forcibly collapsed against the tool shaft 120, the seal structure may be also collapsed and fold down to a smaller size.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A temporary vascular check valve comprising
a central shaft;
a collar positioned about the shaft;
an annular expandable seal structure positioned about the shaft;
a plurality of struts each pivotally attached at a first end to the collar and each attached at a second end to the annular seal structure; and
a plurality of leaflets positioned between each of the plurality of struts, wherein each of the leaflets is connected along lateral edges to adjacent struts and along a bottom edge to the collar.

2. The temporary vascular check valve of claim 1, wherein the annular seal structure is a toroidal balloon.

3. The temporary vascular check valve of claim 1, wherein the annular seal structure is an expandable wire hoop.

4. The temporary vascular check valve of claim 3, wherein the wire hoop is formed of shape memory metal.

5. The temporary vascular check valve of claim 1, wherein a top edge of each of the plurality of leaflets is free and extends to a position to interface with an inner wall of the annular expandable seal structure.

6. The temporary vascular check valve of claim 1, wherein the central shaft is a hollow tube.

7. The temporary vascular check valve of claim 1 further comprising a plurality of mesh segments positioned between each of the plurality of struts and outside the leaflets such that the leaflets are positioned between the mesh segments and the shaft, wherein each of the mesh segments is connected along lateral edges to adjacent struts, along a bottom edge to the collar, and along a top edge to the annular expandable seal structure.

8. The temporary vascular check valve of claim 1 further comprising a control wire connected to the collar and extending proximally along the shaft.

9. The temporary vascular check valve of claim 1 further comprising a fluid injection tube fluidly connected with the annular expandable seal structure and extending proximally along the shaft.

10. A method of inserting a temporary check valve in a vascular structure comprising inserting a tubular shaft within a vascular structure;
advancing the temporary check valve along the tubular shaft to a desired position within the vasculature, wherein the temporary check valve further comprises
a collar positioned about the tubular shaft;
an annular expandable seal structure positioned about the shaft;
a plurality of struts each pivotally attached at a first end to the collar and each attached at a second end to the annular seal structure; and
a plurality of leaflets positioned between each of the plurality of struts, wherein each of the leaflets is connected along lateral edges to adjacent struts and along a bottom edge to the collar; and
expanding the expandable seal structure to seal against sidewalls of the vascular structure.

11. The method of claim 10 further comprising orienting the temporary check valve such that the collar is anterior to the annular expandable seal structure with respect to a direction of vascular fluid flow.

12. The method of claim 10, wherein the operation of inserting further comprises collapsing the expandable seal structure, the leaflets, and the struts to a position against and substantially parallel to the tubular shaft.

13. The method of claim 10, wherein the operation of expanding further comprises manually expanding the struts from a collapsed position against the tubular shaft to seal the expandable seal structure against the sidewalls of the vascular structure.

14. The method of claim 10, wherein the vascular structure is a heart and the operation of inserting further comprises
incising an apex of the heart to access a ventricular chamber; and
inserting the tubular shaft into the ventricular chamber.

15. The method of claim 13, wherein
the operation of advancing further comprises
advancing the tubular shaft through the ventricular chamber, past the aortic valve, and into the aorta; and
positioning the temporary check valve within the ventricular chamber proximal to the aortic valve; and
the operation of expanding further comprises expanding the expandable seal structure to seal against sidewalls of the ventricular chamber proximal to the aortic valve.

16. The method of claim 15, wherein the operation of advancing further comprises positioning the expandable seal structure against an underside of an existing in vivo, in situ bioprosthetic valve.

17. The method of claim 10, wherein
the operation of advancing the temporary check valve further comprises
advancing the tubular shaft through the aorta, past the aortic valve, and into the ventricular chamber; and
positioning the temporary check valve within the ventricular chamber proximal to the aortic valve; and
the operation of expanding further comprises expanding the expandable seal structure to seal against sidewalls of the ventricular chamber proximal to the aortic valve.

18. The method of claim 17, wherein the operation of advancing further comprises positioning the expandable seal structure against an underside of an existing in vivo, in situ bioprosthetic valve.

19. The method of claim 10 further comprising moving the tubular shaft through the collar while retaining the collar in a fixed position in vivo.
